(19) United States
(12) Patent Application Publication
(10) Pub. No.: US 2020/0397499 A1
(43) Pub. Date: Dec. 24, 2020

(54) ELECTRIC SURGICAL INSTRUMENT AND COVER

(71) Applicant: MEDICAROID CORPORATION, Kobe-shi (JP)

(72) Inventor: Kaoru TAKAHASHI, Kobe-shi (JP)

(73) Assignee: MEDICAROID CORPORATION, Kobe-shi (JP)

(21) Appl. No.: 16/862,567

(22) Filed: Apr. 30, 2020

(30) Foreign Application Priority Data
Jun. 21, 2019 (JP) 2019-115176

(51) Int. Cl. A61B 18/14; A61B 34/30; A61B 34/37; A61B 34/00

(52) U.S. Cl. CPC A61B 18/14; A61B 34/30; A61B 2017/00973; A61B 34/74; A61B 34/37

(57) ABSTRACT

An electric surgical instrument to be detachably connected to a robot arm of a robotic surgical system according to one or more embodiments may include: a shaft; an end effector provided at a distal end side of the shaft via a wrist structure; a connection part which is provided at a proximal end side of the shaft and is to be connected to the robot arm; and a cover including an elongated hollow structure to accommodate therein the wrist structure and containing therein an electrical insulation material and an X-ray contrast agent.

### CROSS-REFERENCE TO RELATED APPLICATIONS

This application claims priority based on 35 USC 119 from prior Japanese Patent Application No. 2019-115176 filed on Jun. 21, 2019, entitled “ELECTRIC SURGICAL INSTRUMENT AND COVER”, the entire contents of which are incorporated herein by reference.

### BACKGROUND

This disclosure may relate to an electric surgical instrument and a cover and specifically may relate to an electric surgical instrument detachably attached to a robot arm of a robotic surgical system and a cover of the electric surgical instrument.

In a related art, an electric surgical instrument detachably attached to a robot arm of a robotic surgical system is known (see for example, Patent Document 1).

Patent Document 1 discloses an electric surgical instrument detachably attached to a robot manipulator (a robot arm) of a robotic surgical system. The electric surgical instrument includes an end effector and a wrist structure for operating the end effector. A part of the end effector and the wrist structure are accommodated in an electrical insulation cover. The cover is formed of silicone with excellent flexibility and resilience.
Patent Document 1: JP2013-530771A

### SUMMARY

Although the cover disclosed in Patent Document 1 is formed of silicone with excellent flexibility and resilience, it might be possible that the cover comes off inside a patient's body during surgery due to damages of the cover or the like.

An object of an aspect of one or more embodiments is to provide an electric surgical instrument with a cover which can be searched from a patient's body, even when the cover falls off from the electric surgical instrument inside the patient's body during surgery, and to provide the cover.

A first aspect of one or more embodiments may be an electric surgical instrument detachably attached to a robot arm of a robotic surgical system. The electric surgical instrument may include: a shaft; an end effector provided at a distal end side of the shaft via a wrist structure; a connection part which is provided at a proximal end side of the shaft and is to be connected to the robot arm; and a cover including an elongated hollow structure to accommodate therein the wrist structure and containing therein an electrical insulation material and an X-ray contrast agent. According to the first aspect, the electric surgical instrument includes the cover including the elongated hollow structure to accommodate therein the wrist structure and containing therein the electrical insulation material and the X-ray contrast agent. Accordingly, the cover containing the X-ray contrast agent can be detected by X-ray. Thus, the first aspect can provide the electric surgical instrument with the cover which can be searched from a patient's body, even if the cover comes off from the electric surgical instrument inside the patient's body.

A second aspect of one or more embodiments may be a cover for an electric surgical instrument to be detachably connected to a robot arm of a robotic surgical system. The electric surgical instrument may include: a shaft; an end effector provided at a distal end side of the shaft via a wrist structure; and a connection part which is provided at a proximal end side of the shaft and is to be connected to the robot arm. The cover may include: an electrical insulation material; and an X-ray contrast agent, wherein the cover includes an elongated hollow structure to accommodate therein the wrist structure.

According to the second aspect, the cover includes the elongated hollow structure to accommodate therein the wrist structure and contains therein the electrical insulation material and the X-ray contrast agent. Therefore, like the first aspect related to the electric surgical instrument, the second aspect can provide the cover which can be searched from a patient's body, even if the cover comes off from the electric surgical instrument inside the patient's body.

This disclosure may be able to provide an electric surgical instrument with a cover which can be searched from a patient's body, even if the cover comes off from the electric surgical instrument inside the patient's body during surgery, and may be able to provide such a cover.

### BRIEF DESCRIPTION OF DRAWINGS

FIG. 1 is a diagram illustrating an overview of a robotic surgical system according to first and second embodiments.

FIG. 2 is a block diagram illustrating a view of a control-related configuration of the robotic surgical system according to first and second embodiments.

FIG. 3 is a diagram illustrating a perspective view of a state where an electric surgical instrument is attached to a drive part of a robot arm via an adaptor according to first and second embodiments.

FIG. 4 is a diagram illustrating a perspective view of a state where the surgical instrument and the adaptor are detached from the drive part of the robot arm according to first and second embodiments.

FIG. 5 is a diagram illustrating a perspective view of the electric surgical instrument and the adaptor as viewed from the Z-side according to first and second embodiments.

FIG. 6A is a diagram illustrating a view of a state where a cover of the electric surgical instrument is detached according to a first embodiment.

FIG. 6B is a diagram illustrating a view of a state where the cover of the electric surgical instrument is attached according to a first embodiment.

FIG. 7 is a diagram illustrating a perspective view of the cover of the electric surgical instrument according to a first embodiment.

FIG. 8 is a diagram illustrating a cross sectional view of the cover of the electric surgical instrument according to a first embodiment

### DETAILED DESCRIPTION

Descriptions are provided hereinbelow for one or more embodiments based on the drawings. In the respective drawings referenced herein, the same constituents are designated by the same reference numerals and duplicate explanation concerning the same constituents is omitted. All of the drawings are provided to illustrate the respective examples only.

### First Embodiment

(Configuration of Robotic Surgical System)

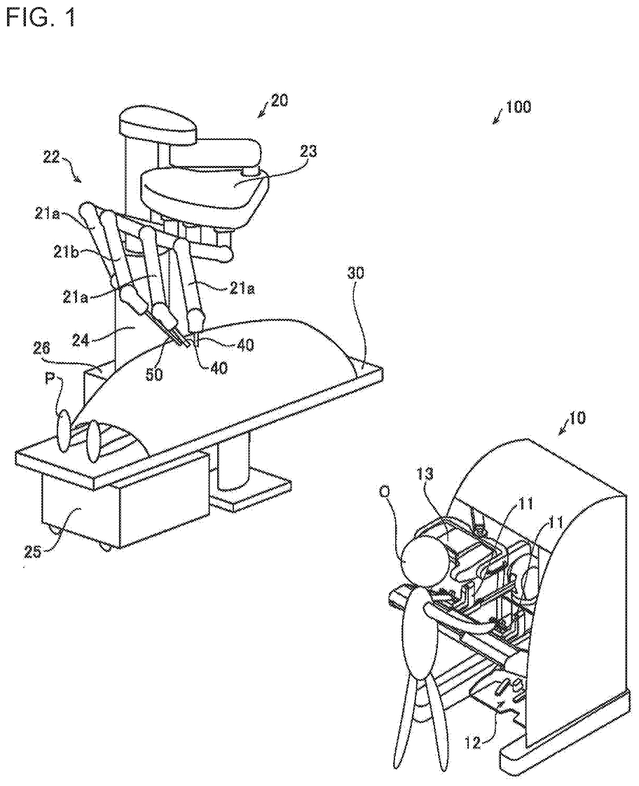

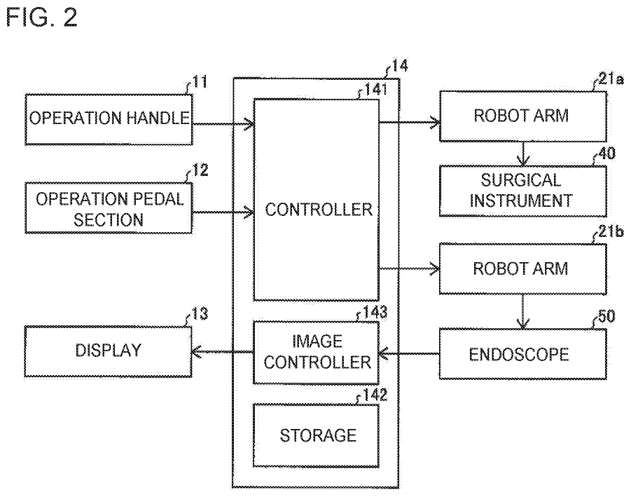

The configuration of a robotic surgical system 100 according to a first embodiment is described with reference to FIGS. 1 and 2.

As illustrated in FIG. 1, the robotic surgical system 100 includes a remote control apparatus 10 and a patient-side apparatus 20. The remote control apparatus 10 is provided to remotely control medical equipment provided for the patient-side apparatus 20. When an operator O, as a surgeon, inputs an action mode instruction to be executed by the patient-side apparatus 20, to the remote control apparatus 10, the remote control apparatus 10 transmits the action mode instruction to the patient-side apparatus 20 via a controller 26. In response to the action mode instruction transmitted from the remote control apparatus 10, the patient-side apparatus 20 operates medical equipment, including surgical instruments 40 attached to robot arms 21a and an endoscope 50 attached to a robot arm 21b. This allows for minimally invasive surgery.

The patient-side apparatus 20 constitutes an interface to perform a surgery for a patient P. The patient-side apparatus 20 is positioned beside an operation table 30 on which the patient P is laid. The patient-side apparatus 20 includes plural robot arms 21a and 21b, to one 21a of which the endoscope 50 is attached and to the others 21b of which the electric surgical instruments 40 are attached respectively. The robot arms 21a and 21b are commonly supported by a platform 23.

Each of the plural robot arms 21a and 21b includes plural joints. Each joint includes a driver provided with a servomotor and a position detector such as an encoder. The robot arms 21a and 21b are configured so that the medical equipment attached to each robot arm 21a or 21b is controlled by a driving signal given through the controller 26 and performs a desired movement.

The platform 23 is supported by the positioner 22 mounted on a floor of an operating room. The positioner 22 includes a column 24 including an elevating shaft adjustable in the vertical direction and a base 25 including wheels and thus being movable on the floor.

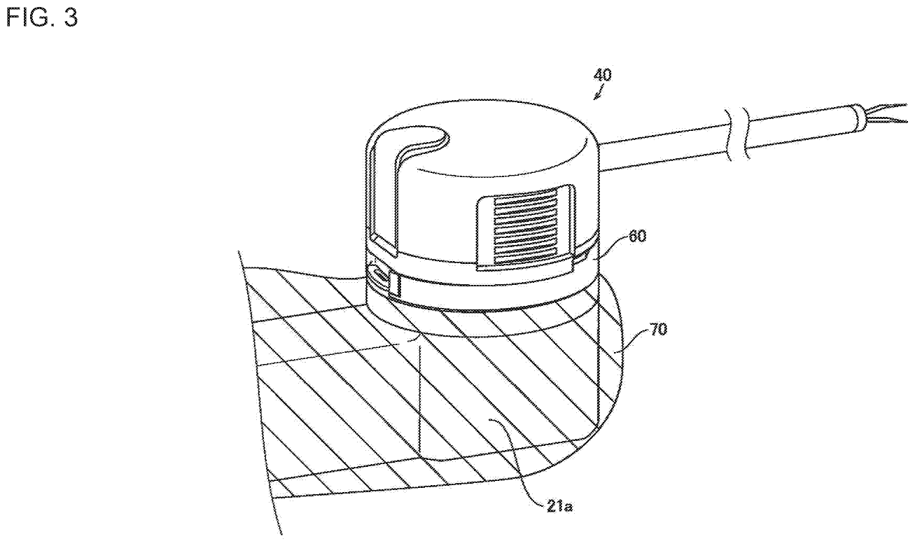

The electric surgical instruments 40 as medical equipment are detachably attached to the distal end portions of the robot arms 21a. Each electric surgical instrument 40 includes: a housing 43 (see FIG. 3) attached to the robot arm 21a; an elongated shaft 42 (see FIG. 3); and an end effector 41 (see FIG. 3) provided at the distal end portion of the shaft 42. The end effector 41 may be grasping forceps, scissors, a hook, a high-frequency knife, a snare wire, a clamp, or a stapler, for example. The end effector 41 is not limited to those and can be various types of treatment tools. In surgeries using the patient-side apparatus 20, the robot arms 21a introduce the electric surgical instruments 40 into the body of the patient P through a cannula (trocar) placed on the body surface of the patient P. The end effector 41 of the electric surgical instrument 40 is then located near the surgery site.

To the distal end portion of the robot arm 21b, the endoscope 50 as the medical equipment is detachably attached. The endoscope 50 captures an image within the body cavity of the patient P. The captured image is outputted to the remote control apparatus 10. The endoscope 50 is a 3D endoscope capable of capturing a three-dimensional image or a 2D endoscope. In surgeries using the patient-side apparatus 20, the robot arm 21b introduces the endoscope 50 into the body of the patient P through a trocar placed on the body surface of the patient P. The endoscope 50 is then located near the surgery site.

The remote control apparatus 10 constitutes the interface with the operator O. The remote control apparatus 10 is an apparatus that allows the operator O to operate the medical equipment attached to the robot arms 21a and 21b. Specifically, the remote control apparatus 10 is configured to transmit action mode instructions which are inputted by the operator O and are to be executed by the electric surgical instruments 40 and endoscope 5, to the patient-side apparatus 20 through the controller 26. The remote control apparatus 10 is installed beside the operation table 30 so that the operator O can see the condition of the patient P very well while operating the remote control apparatus 10, for example. The remote control apparatus 10 may be configured to transmit action mode instructions wirelessly and installed in a room different from the operation room where the operation table 30 is installed.

The action modes to be executed by the electric surgical instruments 40 include modes of actions to be taken by each electric surgical instrument 40 (a series of positions and postures) and actions to be executed by the function of each electric surgical instrument 4. When the electric surgical instrument 40 is a pair of grasping forceps, for example, the action modes to be executed by the electric surgical instrument 40 include roll and pitch positions of the wrist of the end effector 41 and actions to open and close the jaws. When the electric surgical instrument 40 is a high-frequency knife, the action modes to be executed by the electric surgical instrument 40 may include vibration of the high-frequency knife, specifically, supply of current to the high-frequency knife. When the electric surgical instrument 40 is a snare wire, the action modes to be executed by the electric surgical instrument 40 may include a capturing action and an action to release the captured object. Further the action modes may include an action to supply current to a bipolar or monopolar instrument to burn off the surgery site.

The action modes to be executed by the endoscope 50 include, for example, an action mode to set the position and posture of the distal end portion of the endoscope 50 and/or an action mode to set the zoom magnification, for example.

As illustrated in FIGS. 1 and 2, the remote control apparatus 10 includes operation handles 11, an operation pedal section 12, a display part 13, and a control apparatus 14.

The operation handles 11 are provided in order to remotely operate medical equipment attached to the robot arms 21a and 21b. Specifically, the operation handles 11 accept operations by the operator O for operating the medical equipment (electric surgical instruments 40 and the endoscope 5). The operation handles 11 include two operation handles 11 arranged side by side in the horizontal direction. One of the two operation handles 11 is operated by the right hand of the operator O while the other operation handle 11 is operated by the left hand of the operator O.

The operation handles 11 extend from the rear side of the remote control apparatus 10 toward the front side. The operation handles 11 are configured to move in a predetermined three-dimensional operation region. Specifically, the operation handles 11 are configured so as to move up and down, right and left, and forward and rearward.

The remote control apparatus 10 and patient-side apparatus 20 constitute a master-slave system in terms of controlling movement of the robot arms 21a and 21b. The operation handles 11 constitute an operating part on the master side in the master-slave system. The robot arms 21a and 21b holding medical equipment constitute an operating section on the slave side. When the operator O operates the operation handles 11, the movement of the robot arms 21a or 21b is controlled so that the distal end portions (the end effectors 41 of the electric surgical instruments 4) of the robot arms 21a or the distal end portion (the endoscope 5) of the robot arm 21b moves following the movement of the operation handles 11.

The patient-side apparatus 20 controls the movement of the robot arms 21a in accordance with the set motion scaling ratio. When the motion scaling ratio is set to 1/2, for example, the end effectors 41 of the electric surgical instruments 40 move 1/2 of the movement distance of the operation handles 11. This allows for precise fine surgery.

The operation pedal section 12 includes plural pedals to execute medical equipment-related functions. The plural pedals include a coagulation pedal, a cutting pedal, a camera pedal, and a clutch pedal. The plural pedals are operated by a foot of the operator O.

The coagulation pedal enables the electric surgical instrument 40 to coagulate a surgery site. Specifically, when the coagulation pedal is operated, voltage for coagulation is applied to the electric surgical instrument 40 to coagulate a surgery site. The cutting pedal enables the electric surgical instrument 40 to cut a surgery site. Specifically, the cutting pedal is operated to apply voltage for cutting to the electric surgical instrument 40 and cut a surgery site.

The camera pedal is used to control the position and orientation of the endoscope 50 that captures images within the body cavity. Specifically, the camera pedal enables operation of the endoscope 50 by the operation handles 11. The position and orientation of the endoscope 50 are controllable by the operation handles 11 while the camera pedal is being pressed. The endoscope 50 is controlled by using both of the right and left operation handles 11, for example. Specifically, when the operator O rotates the right and left operation handles 11 about the middle point between the right and left operation handles 11, the endoscope 50 is rotated. When the operator O presses the right and left operation handles 11 together, the endoscope 50 goes forward into the body cavity. When the operator O pulls the right and left operation handles 11 together, the endoscope 50 goes back. When the operator O moves the right and left operation handles 11 together up, down, right, or left, the endoscope 50 moves up, down, right, or left, respectively.

The clutch pedal is used to temporarily disconnect operation-related connection between the operation handles 11 and the robot arms 21a and 21b to stop movement of the electric surgical instruments 4. Specifically, when the clutch pedal is being pressed, the robot arms 21a and 21b of the patient-side apparatus 20 do not work even if the operation handles 11 are operated. For example, when the operation handles 11 are operated and moved to the edge of the range of movement, the operator O operates the clutch pedal to temporarily disconnect the operation-related connection and then returns the operation handles 11 to the center of the range of movement. When the operator O stops operating the clutch pedal, the operation handles 11 are again connected to the robot arms 21a and 21b. The operator O restarts the operation for the operation handles 11 around the center thereof.

The display part 13 or a display is configured to display images captured by the endoscope 5. The display part 13 includes a scope type display section or a non-scope type display section. The scope type display section is a display section that the operator O looks into. The non-scope type display section is a display section like an open-type display section that includes a flat screen and the operator O is able to see without looking into, such as normal displays for personal computers.

When the scope type display section is attached, the scope type display section displays 3D images captured by the endoscope 50 attached to the robot arm 21b of the patient-side apparatus 20. When the non-scope type display section is attached, the non-scope type display section also displays 3D images captured by the endoscope 50 provided for the patient-side apparatus 20. The non-scope type display section may display 2D images captured by the endoscope 50 provided for the patient-side apparatus.

As illustrated in FIG. 2, the control apparatus 14 includes a controller 141, a storage 142, and an image controller 143, for example. The controller 141 includes a calculator such as a CPU. The storage 142 includes a memory, such as a ROM (Read Only Memory) and a RAM (Random Access Memory). The control apparatus 14 may be composed of a single controller performing centralized control or may be composed of plural controllers that perform decentralized control in cooperation with each other. The controller 141 determines whether an action mode instruction inputted by the operation handles 11 is to be executed by the robot arm 21a or to be executed by the endoscope 5, depending on the state of the operation pedal section 12. When determining that the action mode instruction inputted by the operation handles 11 is to be executed by any one of the electric surgical instruments 4, the controller 141 transmits the action mode instruction to the corresponding robot arm 21a. The robot arm 21a is thereby driven for controlling movement of the electric surgical instrument 40 attached to the robot arm 21a.

When determining that the action mode instruction inputted by the operation handles 11 is to be executed by the endoscope 5, the controller 141 transmits the action mode instruction to the robot arm 21b. The robot arm 21b is thereby driven for control of movement of the endoscope 50 attached to the robot arm 21b.

The storage 142 stores control programs corresponding to the types of the electric surgical instrument 4, for example. The controller 141 reads the stored control programs according to the types of the attached electric surgical instruments 4. The action mode instructions from the operation handles 11 and/or the operation pedal section 12 of the remote control apparatus 10 thereby causes the respective electric surgical instruments 40 to perform proper movements.

The image controller 143 transmits images acquired by the endoscope 50 to the display part 13. The image controller 143 performs processing and alternations for the images when needed.

(Configuration of Electric Surgical Instrument, Adaptor, Drape, and Robot Arm)

With reference to FIGS. 3 to 5, the configurations of the electric surgical instrument 40, an adaptor 60, a drape 70, and the robot arm 21a according to a first embodiment are described.

As illustrated in FIGS. 3 to 5, the electric surgical instrument 40 is detachably connected to the robot arm 21a through the adaptor 60.

The electric surgical instrument 40 includes a connection part 40a or a connector part, which is an attachment surface of the electric surgical instrument provided at the Z2 side of the housing 43, such that the adaptor 60 is to be attached and connected to the connection part 40a of the electric surgical instrument 40. The connection part 40a of the electric surgical instrument 40 is provided at a proximal end side (the Y2 side) of the shaft 42 and is to be attached and connected to the robot arm 21a via the adaptor 60. The adaptor 60 includes a connection part 60a or a connector part, which is an attachment surface of the adaptor 60 provided at the Z1 side of the adaptor 60, such that the electric surgical instrument 40 is to be attached and connected to the connection part 60a of the adaptor 60. The adaptor 60 also includes a connection part 60b or a connector part, which is an attachment surface of the adaptor 60 provided at the Z2 side of the adaptor 60, such that the adaptor 60 is to be attached and connected, at the connection part 60b, to a drive part 200 of the robot arm 21a. The drive part 200 of the robot arm 21a includes a connection part 21c or a connector part, which is an attachment surface of the drive part 200 provided at the Z1 side of the drive part 200, such that the adaptor 60 is to be attached to the connection part 21c of the drive part 200.

The robot arm 21a is used in a clean area and is thus covered with the drape 70. In operation rooms, clean technique is used in order to prevent surgical incision sites and medical equipment from being contaminated by pathogen, foreign matters, or the like. The clean technique defines a clean area and a contaminated area, which is other than the clean area. The surgery sites are located in the clean area. Members of the surgical team, including the operator O, make sure that only sterile objects are placed in the clean area during surgery and perform sterilization for an object which is to be moved to the clean area from the contaminated area. Similarly, when the members of the surgical team including the operator O, place their hands in the contaminated area, the members sterilize their hands before directly touching objects located in the clean area. Instruments used in the clean area are sterilized or are covered with the drape 70 sterilized.

The drape 70 includes a body section 71 that covers the robot arm 21a and an attachment section 72 sandwiched between the drive part 200 of the robot arm 21a and the adaptor 60. The body section 71 is made of a flexible film member. The flexible film member is made of a resin material, such as thermoplastic polyurethane and polyethylene. The body section 71 includes an opening so that the drive part 200 of the robot arm 21a is engaged with the adaptor 60. In the opening of the body section 71, the attachment section 72 is provided to close the opening. The attachment section 72 is made of a resin mold member. The resin mold member is made of a resin member such as polyethylene terephthalate. The attachment section 72 is harder (less flexible) than the body section 71. The attachment section 72 includes an opening so that the drive part 200 of the robot arm 21a is engaged with the adaptor 60. The opening of the attachment section 72 may be provided corresponding to the section where the drive part 200 of the robot arm 21a is engaged with the adaptor 60. The opening of the attachment section 72 may include plural openings corresponding to plural sections at which the drive part 200 of the robot arm 21a is engaged with the adaptor 60.

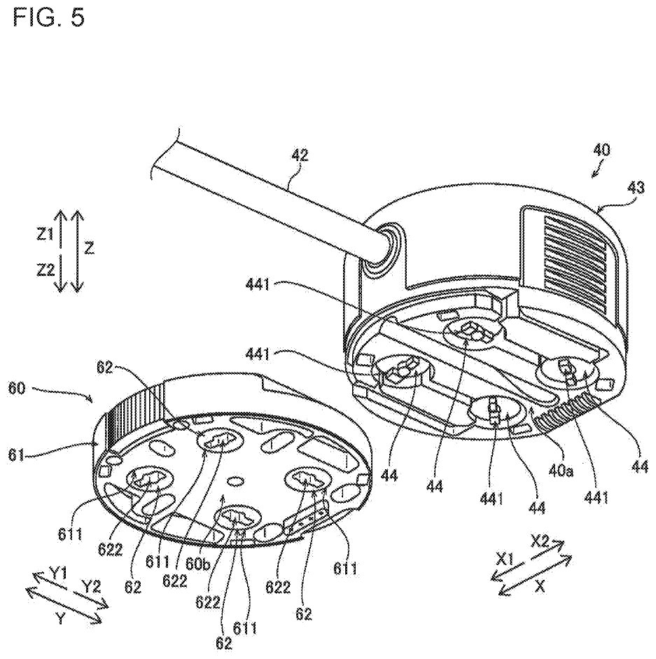

The electric surgical instrument 40 includes plural (four) driven members 44 (see FIG. 5) provided within the housing 43 and being rotatable about the respective rotation axes extending along the Z axis. The plural driven members 44 are provided to operate (drive) the end effector 41. For example, the driven members 44 are connected to the end effector 41 with elongate elements (not illustrated) such as wires and cables inserted through the shaft 42. With this, rotations of the driven members 44 drive the wires, which operate (drive) the end effector 41. In addition, the driven member 44 is connected to the shaft 42 through gears (not illustrated), for example. With this, the shaft 42 is rotated with rotation of the driven member 44.

To transmit driving forces from the drive part 200 of the robot arm 21a, the driven members 44 include fitting projections 441, which are fit to later-described drive transmission members 62 of the adaptor 60. The fitting projections 441 protrude from the Z2-side surfaces of the respective driven members 44 toward the adaptor 60 (in the Z2 direction)

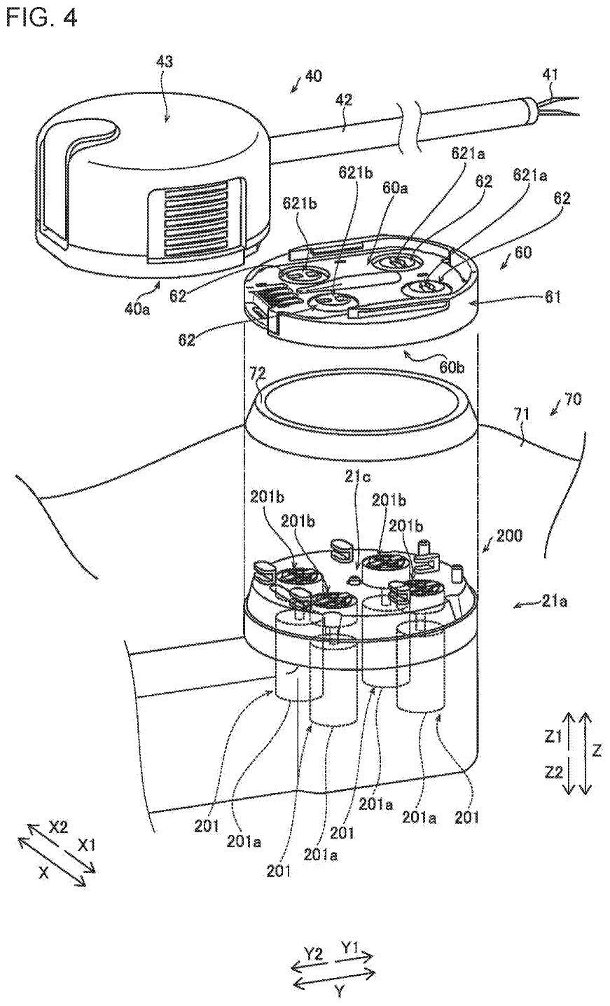

The adaptor 60 includes an adaptor main body 61 and plural (four) drive transmission members 62 supported in the adaptor main body 61 to be rotatable with respect to the adaptor main body 61 about a rotational axis thereof extending along the Z direction. The adaptor main body 61 includes plural (four) through holes 611 penetrating therethrough in the Z direction. In the plural through holes 611, the drive transmission members 62 are respectively provided to be rotatable about the rotational axis thereof. The number of the plural (four) drive transmission members 62 corresponds to the number of the plural (four) the driven members 44 of the electric surgical instrument 40. The drive transmission members 62 are configured to transmit driving forces from the robot arm 21a to the driven members 44 of the electric surgical instrument 40. Each of the drive transmission members 62 includes a fitting recess 621 (see FIG. 4) to be fitted to the fitting projection 441 of the corresponding driven member 44 of the electric surgical instrument 40. The fitting recess 621 is formed at a portion of the drive transmission member 62 on the side of the electric surgical instrument 40 (the Z1 side), such that the fitting recess 621 is recessed from the Z1 side surface of the drive transmission member 62 toward the side (the Z2 side) opposite to the electric surgical instrument 40 side.

Each of the drive transmission members 62 also includes a fitting recess 622 (see FIG. 5) to be fitted to a fitting projection 201b of a corresponding one of later-described drive members 201 of the drive part 200 of the robot arms 21a. The fitting recess 622 is formed at a portion of the drive transmission member 62 on the side of the robot arm 21a, such that the fitting recess is recessed from the Z2 side surface of the drive transmission member 62 toward the side (the Z1 side) opposite to the robot arm 21a side.

The robot arm 21a includes the drive part 200 to drive the driven members 44 of the electric surgical instrument 40. The drive part 200 of the robot arm 21a generates the driving force to be applied to the driven members 44 of the electric surgical instrument 40. Specifically, the drive part 200 includes a plurality (four) of the drive members 201 which are provided to correspond to the driven members 44 of the electric surgical instrument 40. Each of the drive members 201 includes an actuator 201a including a motor serving as a driving source and the fitting projection 201b to be rotated by the actuator 201a about the rotational axis thereof extending the Z direction. The fitting projection 201b is projected from the Z1 side surface of the drive member 201 toward the adaptor 60 side (the Z1 side).

(Configuration of Cover of Electric Surgical Instrument)

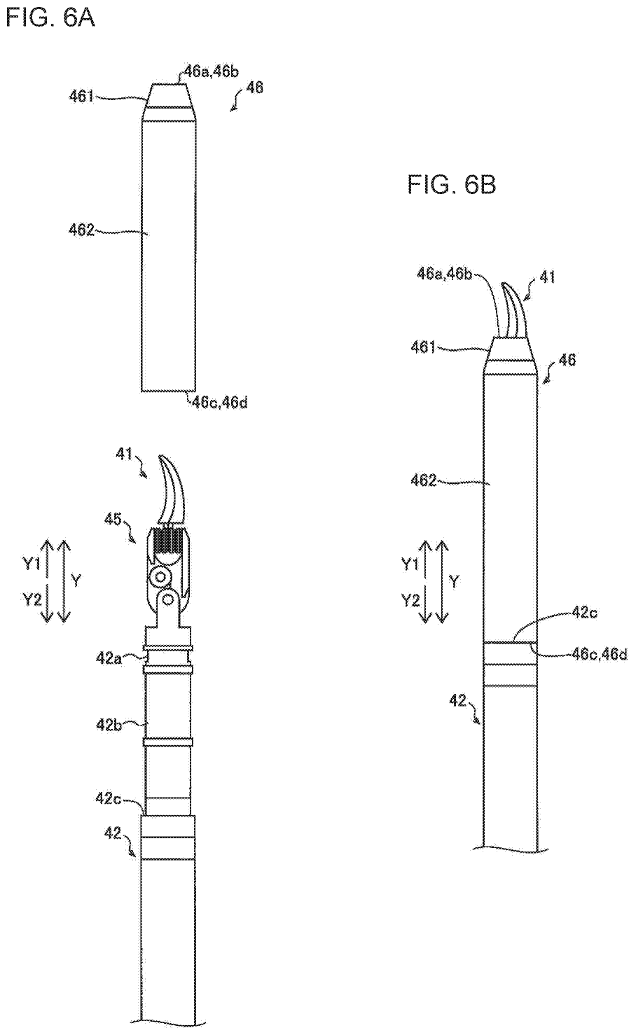

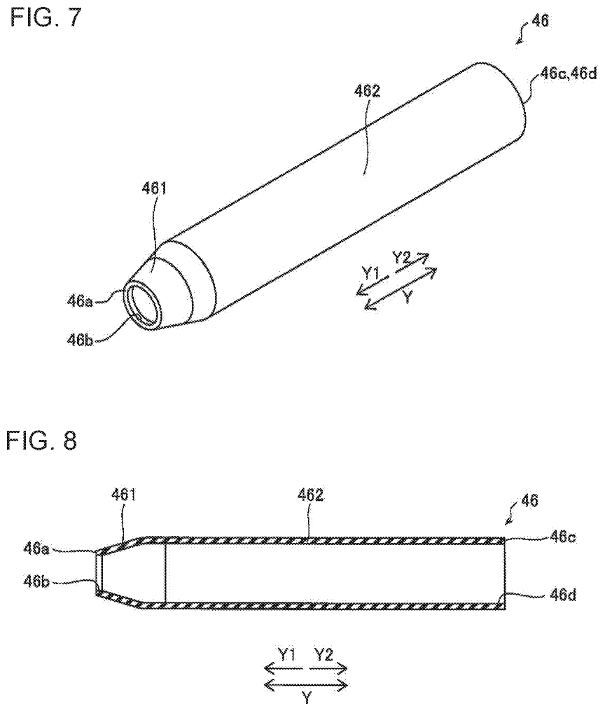

With reference to FIGS. 6A to 8, the configuration of a cover 46 of the electric surgical instrument 40 is described below.

As illustrated in FIGS. 6A and 6B, the end effector 41 is provided at a distal end side of the shaft 42 via a wrist structure 45. The wrist structure 45 is a structure provided at the distal end portion 42a of the shaft 42 for an operation of changing the position and posture of the end effector 41. The wrist structure 45 is for example configured to allow the end effector 41 to be rotatable about the pitch axis.

The end effector 41 may be an electrocautery end effector that cauterizes a living tissue of the patient P by heat generated by electric energy. The electrocautery end effector can be, for example, a monopolar end effector, a bipolar end effector, and the like. High-frequency AC power can be supplied to the electrocautery end effector as electric energy from a power supply source (not shown).

In a case where the end effector 41 is an electrocautery end effector, the cover 46 (insulation cover) having an electrical insulation property is used to suppress conduction of the electric energy to the living tissue of the patient P at an unintended position. The cover 46 is detachably connected to the shaft 42.

The cover 46 has an elongated hollow structure to accommodate therein at least a part of the end effector 41 and the wrist structure 45. The cover 46 includes an electrical insulation material. The electrical insulation material has enough flexibility to allow the end effector 41 to be operated (changed in the posture) with the wrist structure 45 being accommodated in the cover 46. Thus, the electrical insulation material does not hinder the operation of the end effector 41 so that the end effector 41 can be smoothly operated. The electrical insulation material may be an elastically deformable material such as rubber. For example, the electrical insulation material may be silicone (silicone rubber). Thus, the cover 46 can be made using silicone that has flexibility, resilience, high electrical insulation, and high temperature resistance. Specifically, in view of smooth operation of the end effector 41, it is preferable to use silicone whose elongation according to DIN53504-S1 has a ratio of 100% or more and 1000% or less, and more preferably 500% or more and 900% or less. Also, in view of wearability of the cover 46 to the electric surgical instrument 40, it is preferable to use silicone whose hardness according to DIN 53505 (Shore A) has 50 or more and 70 or less. Examples of such silicone may include SILPURAN R 8020/60 and SILPURAN R 8030/60 manufactured by Asahi Kasei Wacker Silicone Co., Ltd., or the like.

In a first embodiment, the cover 46 contains an X-ray contrast agent. The X-ray agent may be a medical agent containing a material through which X rays are difficult to transmit. Accordingly, the cover 46 containing the X-ray contrast agent can be detected by the X-rays. Therefore, even if the cover 46 comes off from the electric surgical instrument 40 inside the patient's body during surgery, the cover 46 can be searched out from the patient's body.

The X-ray contrast agent may be, for example, barium sulfate, a bismuth compound such as bismuth oxide, lead oxide, calcium carbonate, potassium titanate, an iodine compound and the like. Preferably, the X-ray contrast agent may be barium sulfate or the like having excellent electric insulation. Further, the mixing ratio of the X-ray contrast agent may be, for example, 5% by weight or more and 50% by weight or less, preferably 15% by weight or more and 45% by weight or less, and more preferably 20% by weight or more and 35% by weight or less. The X-ray contrast agent is evenly dispersed from the distal end (one end) to the proximal end (the other end) of the cover 46. Specifically, the cover 46 is composed of a material obtained by uniformly mixing the X-ray contrast agent with the electrical insulation material.

As illustrated in FIG. 6A to 8, the cover 46 includes an opening 46b at a distal end portion 46a of the cover 46, with the size of the opening 46b being large enough to insert the end effector 41 through. The opening 46b has a substantially circular shape. The cover 46 also includes an opening 46d at a proximal end portion 46c of the cover 46, with the size of the opening 46d being large enough to insert the end effector 41, the wrist structure 45, and a distal end portion 42d of the shaft 42. The opening 46c has a substantially circular shape. The diameter of the opening 46d is larger than the diameter of the opening 46b.

The cover 46 is configured such that the end effector 41 is inserted into the cover 46 from the opening 46d. Accordingly, the cover 46 is configured to be attached to the shaft 42. Specifically, the cover 46 is configured such that the end effector 41, the wrist structure 45, and the distal end portion 42d of the shaft 42 are inserted into the cover 46 until the proximal end portion 46c having the opening 46d is stopped by a cover stopper part 42c, which a step portion of the shaft 42 opposed to the proximal end of the cover 46. That is, the cover 46 is configured to accommodate therein a part of the end effector 41, the wrist structure 45, and the distal end portion 42d of the shaft 42 in the state where the cover 46 is stopped by the cover stopper part 42c of the shaft 42. In this state, the remaining part (distal side portion) of the end effector 41 is not accommodated in the cover 46 and exposed from the opening 46b to the outside.

The cover 46 includes a distal side portion 461 and a proximal side portion 462 continuously connected to the distal side portion 461. Note that the cover 46 may be a molded product integrally formed from the distal end portion 46a to the proximal end portion 46c. That is, the distal side portion 461 and the proximal side portion 462 are different portions of one member that is integrally formed by one molding. The distal side portion 461 and the proximal side portion 462 both include the X-ray contrast agent. Thus, even if either of the distal side portion 461 or the proximal side portion 462 of the cover 46 is broken and comes off from the electric surgical instrument 40 inside the body of the patient P, the cover 46 in the patient's body P can be searched out by the X-ray. The X-ray contrast agent is evenly dispersed over the entire of the cover 46 from the distal side portion 461 to the proximal side portion 462.

The distal side portion 461 has a tapered shape (truncated cone shape) whose outer diameter gradually decreases toward the distal end. The distal side portion 461 is configured to accommodate therein at least a part of the end effector 41. The distal side portion 461 includes, at the distal end (the tip) thereof, the distal end portion 46a and the opening 46b. The proximal side portion 462 has a cylindrical shape with substantially constant outer and inner diameters. The proximal side portion 462 is configured to accommodate therein at least the wrist structure 45 and the distal end portion 42b of the shaft 42. The proximal side portion 462 includes, at the proximal end (the base end) thereof, the proximal end portion 46c and the opening 46d.

### Second Embodiment

In a second embodiment, the proximal side portion 462 contains therein an electrical insulation material whose tear strength is higher than that of an electrical insulation material in the distal side portion 461.

The tear strength of the electrical insulation material of the distal side portion 461 may be, for example, not less than 30 KN/m and not more than 60 KN/m. The tear strength of the electrical insulation material of the proximal side portion 462 may be, for example, not less than 60 KN/m and not more than 160 KN/m.

[Modifications]

It should be understood that one or more embodiments described above are illustrated by way of example in every respect and not limit the invention. The scope of the invention is indicated by claims, not by explanation of one or more embodiments described above, and includes equivalents to claims and all alterations (modifications) within the same.

For example, in a first embodiment described above, the X-ray contrast agent is evenly dispersed from the distal end to the proximal end of the cover. However, the invention is not limited to this. For example, in a modification, the X-ray contrast agent does not need to be evenly dispersed from the distal end to the proximal end of the cover, as long as the cover inside the patient's body is detectable.

In a first embodiment described above, the electrical insulation material of the cover is silicone (silicone rubber). However, the invention is not limited to this. In a modification, the electrical insulation material of the cover may be a material other than silicone, such as polyurethane rubber or the like, for example.

In a first embodiment described above, the distal side portion and the proximal side portion of the cover are portions of one member that is integrally formed by one molding. However, the invention is not limited to this. In a modification, the distal side portion and the proximal side portion of the cover may be made from separate members. In this case, the distal side portion and the proximal side portion of the cover may be joined by heat welding or the like such that the distal side portion and the proximal side portion of the cover are integrally formed.

In a first embodiment described above, the electrical insulation material of the distal side portion and the electrical insulation material of the proximal side portion are the same material. However, the invention is not limited to this. In a modification, the electrical insulation material of the distal side portion and the electrical insulation material of the proximal side portion may be different. Further in a modification of a second embodiment, the electrical insulation material of the distal side portion and the electrical insulation material of the proximal side portion may be different such that the tear strength of the electrical insulation material of the proximal side portion is higher than that of the electrical insulation material of the distal side portion.

1. An electric surgical instrument to be detachably connected to a robot arm of a robotic surgical system, comprising:
a shaft;
an end effector provided at a distal end side of the shaft via a wrist structure;
a connection part which is provided at a proximal end side of the shaft and is to be connected to the robot arm; and
a cover including an elongated hollow structure to accommodate therein the wrist structure and containing therein an electrical insulation material and an X-ray contrast agent.

2. The electric surgical instrument according to claim 1, wherein
the cover includes at a distal end portion an opening whose size allows the end effector to be inserted therethrough; and
the electrical insulation material of the cover has flexibility to allow the end effector to be manipulated with the cover accommodating therein the wrist structure.

3. The electric surgical instrument according to claim 1, wherein
the electrical insulation material contains silicone or polyurethane rubber.

4. The electric surgical instrument according to claim 1, wherein
the cover includes a distal side portion and a proximal side portion connected to the distal side portion, and
the proximal side portion of the cover contains the electrical insulation material whose tear strength is higher than that of the electrical insulation material in the distal side portion.

5. The electric surgical instrument according to claim 4, wherein
the distal side portion and the proximal side portion of the cover both contain the X-ray contrast agent.

6. The electric surgical instrument according to claim 1, wherein
the electrical insulation material has an elongation ratio according to DIN53504-S1 of 100% or more and 1000% or less.

7. The electric surgical instrument according to claim 1, wherein
the electrical insulation material has a hardness according to DIN 53505 (Shore A) of 50 or more and 70 or less.

8. The electric surgical instrument according to claim 1, wherein
the cover contains 5% or more and 50% or less by weight of the X-ray contrast agent.

9. The electric surgical instrument according to claim 1, wherein
the cover includes a first opening at a distal end portion thereof and a second opening at a proximal end portion thereof, the second opening being larger than the first opening.

10. The electric surgical instrument according to claim 1, wherein
the electric surgical instrument is a monopolar electric surgical instrument or a bipolar electric surgical instrument.

11. The electric surgical instrument according to claim 1, wherein
the connection part of the electric surgical instrument is connected to a drive part of the robot arm via an adaptor.

12. A cover for an electric surgical instrument to be detachably connected to a robot arm of a robotic surgical system, wherein the electric surgical instrument including: a shaft; an end effector provided at a distal end side of the shaft via a wrist structure; and a connection part which is provided at a proximal end side of the shaft and is to be connected to the robot arm, the cover comprising:
an electrical insulation material; and
an X-ray contrast agent, wherein the cover includes an elongated hollow structure to accommodate therein the wrist structure.

13. The cover according to claim 12, comprising
an opening provided at a distal end portion of the cover and having a size allowing the end effector to be inserted therethrough, wherein
the electrical insulation material of the cover has a flexibility to allow the end effector to be manipulated with the cover accommodating therein the wrist structure.

14. The cover according to claim 12, wherein,
the electrical insulation material contains silicone or polyurethane rubber.

15. The cover according to claim 12, further comprising:
a distal side portion; and
a proximal side portion connected to the distal side portion, wherein
the proximal side portion of the cover contains the electrical insulation material whose tear strength is higher than that of the electrical insulation material of the distal side portion.

16. The cover according to claim 15, wherein
the distal side portion and the proximal side portion both contain the X-ray contrast agent.

17. The cover according to claim 12, wherein
the electrical insulation material has an elongation ratio according to DIN53504-S1 of 100% or more and 1000% or less.

18. The cover according to claim 12, wherein
the electrical insulation material has a hardness according to DIN 53505 (Shore A) of 50 or more and 70 or less.

19. The cover according to claim 12, wherein,
the cover contains 5% or more and 50% or less by weight of the X-ray contrast agent.

20. The cover according to claim 12, wherein
the electric surgical instrument is a monopolar electric surgical instrument or a bipolar electric surgical instrument.
